(19) United States
(12) Patent Application Publication
(10) Pub. No.: US 2025/0009521 A1
(43) Pub. Date: Jan. 9, 2025

(54) STEMLESS IMPLANT ASSEMBLIES WITH SCREW FIXATION

(71) Applicant: ARTHREX, INC., Naples, FL (US)

(72) Inventors: Nick METCALFE, Bonita Springs, FL (US); Kevin John GALLEN, Naples, FL (US)

(21) Appl. No.: 18/218,671

(22) Filed: Jul. 6, 2023

(51) Int. Cl. A61F 2/40; A61F 2/30

(52) U.S. Cl. CPC A61F 2/4003; A61F 2/30749; A61F 2002/30224; A61F 2002/30433; A61F 2002/30477; A61F 2002/30604; A61F 2002/30654; A61F 2002/30774; A61F 2002/30784; A61F 2002/3085; A61F 2002/4022

(57) ABSTRACT

Arthroplasty implant systems and methods are provided for restoring the functionality of a joint. The arthroplasty implant systems may include stemless implant assemblies capable of establishing a stemless convertible platform for interfacing with articular implants. An exemplary stemless implant assembly may include a cup, a first fixation device insertable through a first central bore of the cup, and a second fixation device insertable through a second central bore of the first fixation device. The first fixation device may be a screw or an anti-rotation implant, and the second fixation device may be a screw.

### BACKGROUND

This disclosure relates to the field of arthroplasty, and more particularly to arthroplasty implant systems that include stemless humeral implant assemblies configured for screw fixation.

Many bones of the human musculoskeletal system include articular surfaces. The articular surfaces cooperate to facilitate different types and degrees of joint movement. The articular surfaces can erode or experience bone loss over time due to repeated use or wear, thereby causing joint instability and pain.

Arthroplasty is an orthopedic surgical procedure performed to repair or replace joints that exhibit degenerative bone deficiencies. Bone deficiencies may occur along the articular surfaces of bone. Some arthroplasty procedures utilize one or more implants to repair the articular surfaces.

### SUMMARY

This disclosure relates to arthroplasty implant systems and methods designed for restoring functionality to a joint. The arthroplasty implant systems may include a humeral implant assembly that includes a stemless convertible implant assembly.

An exemplary stemless implant assembly for a humeral implant assembly may include, inter alia, a cup, a first fixation device insertable through a first central bore of the cup, and a second fixation device insertable through a second central bore of the first fixation device.

In any of the foregoing implementations, the first fixation device is a major screw, and the second fixation device is a minor screw.

In any of the foregoing implementations, the first fixation device is an anti-rotation implant, and the second fixation device is a screw.

In any of the foregoing implementations, in an assembled condition of the stemless implant assembly, a first distal section of the first fixation device is a first distance from the cup, and a second distal section of the second fixation device is a second distance from the cup. The second distance is a greater distance than the first distance.

In any of the foregoing implementations, the second distal section of the second fixation device includes a thread.

In any of the foregoing implementations, the first central bore extends between a rim and a rounded base of the cup.

In any of the foregoing implementations, the rim of the cup includes a flange.

In any of the foregoing implementations, the first fixation device includes a thread or a barb.

In any of the foregoing implementations, the second fixation device includes a thread.

In any of the foregoing implementations, the cup includes a cylindrical shaped body having a threaded inner diameter wall and a smooth outer diameter wall.

An exemplary humeral implant assembly for an arthroplasty implant system may include, inter alia, an articular implant, and a stemless implant assembly adapted to establish a convertible platform for receiving the articular implant. The stemless implant assembly includes a cup, a first fixation device insertable through the cup, and a second fixation device insertable through the first fixation device.

In any of the foregoing implementations, the articular implant includes a concave articular surface.

In any of the foregoing implementations, the articular implant includes a convex articular surface.

In any of the foregoing implementations, the cup includes a cylindrical shaped body having a threaded inner diameter wall and a smooth outer diameter wall.

In any of the foregoing implementations, the first fixation device is a screw that includes an outer diameter wall and a thread circumferentially disposed about at least a portion of the outer diameter wall.

In any of the foregoing implementations, the first fixation device is an anti-rotation implant that includes a tapered body having at least two barbs, and each of the at least two barbs includes a ledge extending circumferentially around at least a portion of the tapered body.

In any of the foregoing implementations, the second fixation device is a screw that includes an outer diameter wall and a thread circumferentially disposed about at least a portion of the outer diameter wall.

In any of the foregoing implementations, in an assembled condition of the stemless implant assembly, a first distal section of the first fixation device is a first distance from the cup, and a second distal section of the second fixation device is a second distance from the cup. The second distance is a greater distance than the first distance.

In any of the foregoing implementations, the cup, the first fixation device, and the second fixation device are coaxially arranged along a central longitudinal axis of the stemless implant assembly.

An exemplary surgical method may include, inter alia, preparing a humerus for receiving a cup of a stemless implant assembly, positioning the cup within the prepared humerus, inserting a first fixation device of the stemless implant assembly through the cup and into a cancellous bone of the humerus, and inserting a second fixation device of the stemless implant assembly through the first fixation device and into the cancellous bone. The second fixation device extends further into the cancellous bone than the first fixation device after the inserting.

### BRIEF DESCRIPTION OF THE DRAWINGS

FIG. 1 illustrates a humeral implant assembly of an arthroplasty implant system.

FIG. 2 illustrates another exemplary humeral implant assembly of an arthroplasty implant system.

FIG. 3 is a perspective view of a stemless implant assembly of a humeral implant assembly.

FIG. 4 is a cross-sectional view through section 4-4 of FIG. 3.

FIG. 5 is an exploded view of the stemless implant assembly of FIGS. 3 and 4.

FIG. 6 illustrates an exemplary cup of the stemless implant assembly of FIGS. 3, 4, and 5.

FIG. 7 is a perspective view of another exemplary stemless implant assembly of a humeral implant assembly.

FIG. 8 is a cross-sectional view through section 8-8 of FIG. 7.

FIG. 9 is an exploded view of the stemless implant assembly of FIGS. 7 and 8.

FIG. 10 schematically illustrates an exemplary surgical method for performing a shoulder arthroplasty procedure.

### DETAILED DESCRIPTION

This disclosure describes arthroplasty implant systems and methods for restoring the functionality of a joint. The arthroplasty implant systems may include implants capable of establishing a stemless convertible platform for interfacing with articular implants.

In some implementations, the arthroplasty implant systems of this disclosure may include a stemless implant assembly that includes a cup, a first fixation device insertable through a first central bore of the cup, and a second fixation device insertable through a second central bore of the first fixation device. The first fixation device may be a screw or an anti-rotation implant, and the second fixation device may be a screw. These and other features of this disclosure are further detailed below.

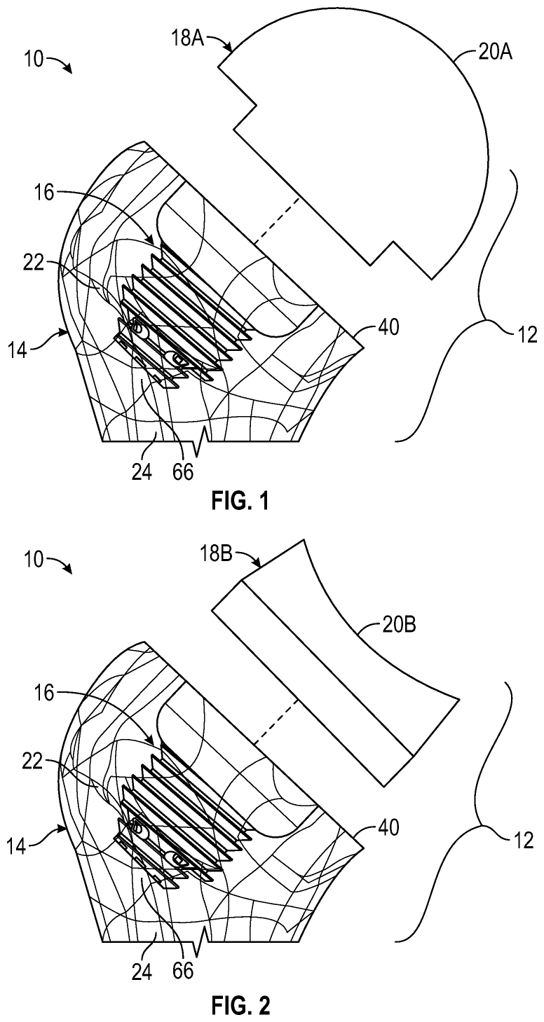

FIG. 1 illustrates an arthroplasty implant system 10 that includes a humeral implant assembly 12. The humeral implant assembly 12 may be implanted within a humerus 14 of a shoulder joint to aid in reconstructing the native articular surface of the humerus 14 and/or restoring the functionality (e.g., range or motion, stability, etc.) of the shoulder joint. Although not shown, the arthroplasty implant system 10 could additionally include a glenoid implant assembly that is configured to interface with the humeral implant assembly 12 for restoring functionality to the shoulder joint. Moreover, although the teachings of this disclosure are described with specific reference to the shoulder joint, this disclosure is not intended to be limited to any particular joint of the human musculoskeletal system and could be applicable to other joints, such as the hip joint, for example.

The humeral implant assembly 12 may include a stemless implant assembly 16 and an anatomic articular implant 18A (see FIG. 1) or a reverse articular implant 18B (see FIG. 2). The anatomic articular implant 18A may be utilized in combination with the stemless implant assembly 16 for performing anatomic total shoulder arthroplasty procedures, and the reverse articular implant 18B may be utilized in combination with the stemless implant assembly 16 for performing reverse shoulder arthroplasty procedures. In reverse shoulder arthroplasty procedures, the reconstituted humerus 14 provides the socket portion and the glenoid (not shown) provides the ball portion of the ball-and-socket joint, which is the opposite of the native anatomy. The anatomic articular implant 18A may include a convex articular surface 20A, and the reverse articular implant 18B may include a concave articular surface 20B. The articular surfaces 20A, 20B are configured to interface with the native glenoid or a glenoid implant assembly of the arthroplasty implant system 10.

In some implementations, the stemless implant assembly 16, the anatomic articular implant 18A, and the reverse articular implant 18B may be provided together as part of a surgical kit. The surgical kit could additionally multiple sizes of each of the stemless implant assembly 16, the anatomic articular implant 18A, and the reverse articular implant 18B.

In the illustrated embodiment, a humeral head of the humerus 14 has been resected, and thus the native articular component of the humerus 14 is removed in order to prepare the humerus 14 for receiving the humeral implant assembly 12. After the humerus 14 has been appropriately prepared, the stemless implant assembly 16 may be secured within a metaphysis 22 of the humerus 14. In an embodiment, the stemless implant assembly 16 is a stemless implant of the humeral implant assembly 12 and therefore lacks a stem that extends into a diaphysis 24 of the humerus 14. The stemless implant assembly 16 may be configured to establish a convertible platform for receiving either the anatomic articular implant 18A or the reverse articular implant 18B. The anatomic articular implant 18A or the reverse articular implant 18B may be mounted to the stemless implant assembly 16 for assembling the humeral implant assembly 12.

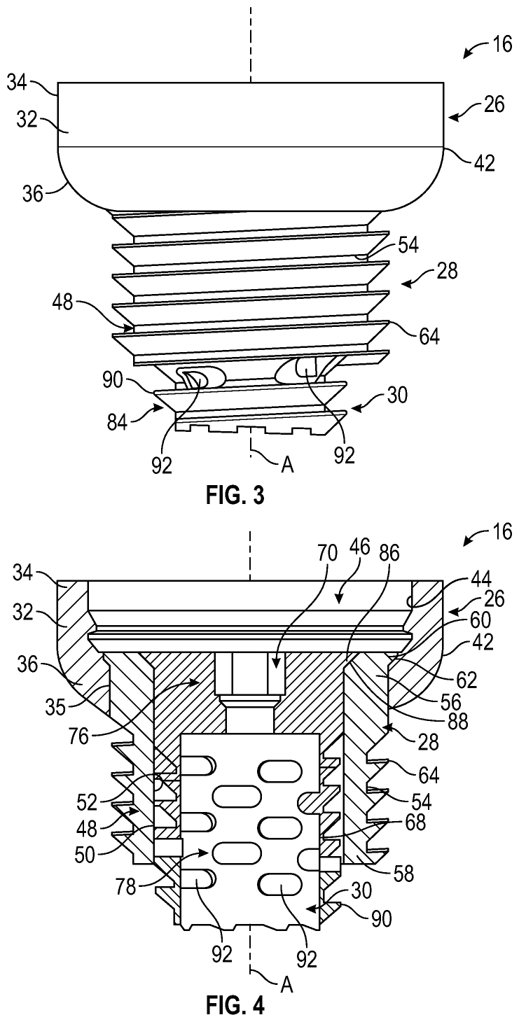

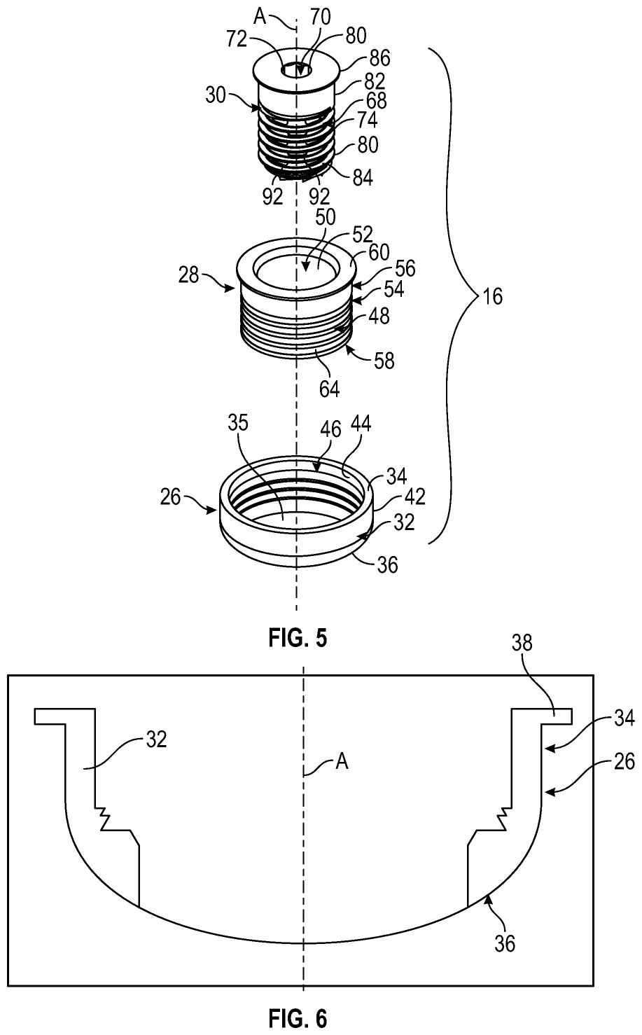

The stemless implant assembly 16 of the humeral implant assembly 12 is further illustrated in FIGS. 3, 4, and 5 (with continued reference to FIGS. 1 and 2). In an embodiment, the stemless implant assembly 16 is constructed of a titanium material. In another embodiment, the stemless implant assembly 16 may be made of a material that has an elastic modulus that is relatively close to that of cortical bone, such as polyether ether ketone (PEEK), for example. Other materials or combinations of materials for constructing the stemless implant assembly 16 could alternatively be utilized within the scope of this disclosure.

The stemless implant assembly 16 may include a cup 26, a first screw 28 (e.g., a first fixation device), and a second screw 30 (e.g., a second fixation device). In this embodiment, the stemless implant assembly 16 embodies a three-piece design. The first screw 28 may be referred to as a major screw of the stemless implant assembly 16, and the second screw 30 may be referred to a minor screw of the stemless implant assembly 16. The first screw 28 and the second screw 30 may additionally be referred to as cage screws.

The cup 26, the first screw 28, and the second screw 30 may be coaxially arranged along a central longitudinal axis A. When the stemless implant assembly 16 is assembled, the first screw 28 may extend at least partially through the cup 26, and the second screw 30 may extend at least partially through the first screw 28.

The cup 26 may include a cylindrical shaped body 32 that extends between a rim 34 located at a top or proximal side of the cylindrical shaped body 32 and a rounded base 36 located at a bottom or distal side of the cylindrical shaped body 32. The rim 34 may thus be located on an opposite side of the cylindrical shaped body 32 from the rounded base 36.

In an embodiment, the rim 34 is flangeless. In another embodiment, the rim 34 includes a flange 38 (see, e.g., FIG. 6). The flange 38 may be configured to load against the cortical rim 40 (see FIG. 1) of the humerus 14 in an implanted position of the stemless implant assembly 16. Engaging the cortical rim 40 in this manner may provide additional fixation support and load the proximal portion of the humerus 14 more favorably. The flange 38 may be either circular or elliptical shaped. However, the actual shape of the flange 38 is not intended to limit this disclosure.

The cylindrical shaped body 32 of the cup 26 may include an outer diameter wall 42 and an inner diameter wall 44. The outer diameter wall 42 may be a relatively smooth surface, and the inner diameter wall 44 may be a threaded surface.

The cylindrical shaped body 32 of the cup 26 may include a central bore 35. The central bore 35 may be a void circumscribed by the inner diameter wall 44. The central bore 35 may be sized and shaped to receive the first screw 28 therethrough.

A receiving cavity 46 of the cup 26 may be configured to receive and secure either the anatomic articular implant 18A or the reverse articular implant 18B to the stemless implant assembly 16. The receiving cavity 46 may extend entirely through the cylindrical shaped body 32.

The receiving cavity 46 may provide an inlay design in which a majority of the stemless implant assembly 16 (with the exception of the flange 38 to the extent it is provided) is disposed inside the humerus 14 post implantation. In this way, the connection between the stemless implant assembly 16 and the articular implant 18A, 18B is also inlaid rather than exhibiting an onlay design.

The first screw 28 may include a cylindrical body 48 that includes a central bore 50. The central bore 50 may be a void circumscribed by an inner diameter wall 52 of the cylindrical body 48. The central bore 50 may be sized and shaped to receive the second screw 30 therethrough.

The cylindrical body 48 of the first screw 28 may additionally include an outer diameter wall 54 having a proximal section 56 and a distal section 58. The proximal section 56 may be a relatively smooth surface that includes a radially extending peripheral lip 60. The peripheral lip 60 may establish a proximal most portion of the proximal section 56. The peripheral lip 60 may be configured to interface with a tapered ledge 62 formed on the inner diameter wall 44 of the cup 26 to prevent over-insertion of the first screw 28 distally through the cup 26.

The distal section 58 of the first screw 28 may include a thread 64 that is circumferentially disposed about a portion of the outer diameter wall 54. The thread 64 may be a self-tapping thread configured to allow the first screw 28 to be screwed into the humerus 14. The thread 64 may be configured such that either a clockwise rotation or a counterclockwise rotation functions to advance the first screw 28 into the humerus 14. In an implanted position of the stemless implant assembly 16, an entirety of the thread 64 is located distally of the cup 26. At this position, the thread 64 is capable of engaging cancellous bone 66 (see FIG. 1) of the humerus 14.

The second screw 30 may include a cylindrical body 68 that includes a central bore 70. The central bore 70 may be a void circumscribed by an inner diameter wall 72 of the cylindrical body 68. The central bore 70 may include a proximal portion 76 and a distal portion 78, which may have a larger diameter than the proximal portion 76. The proximal portion 76 may include a driver recess 80 for receiving a driver instrument that can be used to insert the second screw 30 through the first screw 28.

The cylindrical body 68 of the second screw 30 may additionally include an outer diameter wall 74 having a proximal section 82 and a distal section 84. The proximal section 82 may be relatively smooth and may include a radially extending peripheral lip 86. The peripheral lip 86 may establish a proximal most portion of the proximal section 82. The peripheral lip 86 may be configured to interface with a tapered ledge 88 formed on the inner diameter wall 52 of the first screw 28 to prevent over-insertion of the second screw 30 distally through the first screw 28.

The distal section 84 of the second screw 30 may include a thread 90 that is circumferentially disposed about the distal section 84 of the outer diameter wall 74. The thread 90 may be a self-tapping thread configured to allow the second screw 30 to be screwed into the humerus 14 at a location that is radially inward from the first screw 28. The thread 90 may be configured such that either a clockwise rotation or a counterclockwise rotation functions to advance the second screw 30 into the humerus 14. In an implanted position of the stemless implant assembly 16, a proximal portion of the thread 90 may engage the inner dimeter wall 52 of the first screw 28, and a distal portion of the thread 90 may be located distally of the first screw 28. At this position, the thread 90 is capable of engaging additional cancellous bone 66 (see FIG. 1) of the humerus 14 that is located radially inward of that which is engaged by the thread 64 of the first screw 28. The first screw 28 and the second screw 30 may therefore cooperate to provide dual screw fixation of the cup 26 in this implementation.

The distal section 84 of the second screw 30 may additionally be equipped with a plurality of openings 92. The openings 92 may facilitate bony ingrowth post-insertion of the second screw 30. The openings 92 may be oblong shaped, in an embodiment.

The design characteristics (e.g., thread pitch, thread angle, thread tip width, thread root width, thread depth, thread root radius, etc.) associated with the thread 64 and the thread 90 may vary and are not intended to limit this disclosure. Moreover, the actual dimensions of the cup 26, the first screw 28, and the second screw 30 may vary depending on the size of the patient, among other factors. The surgical kit referenced above could therefore include cups 26 having multiple outer diameter sizes.

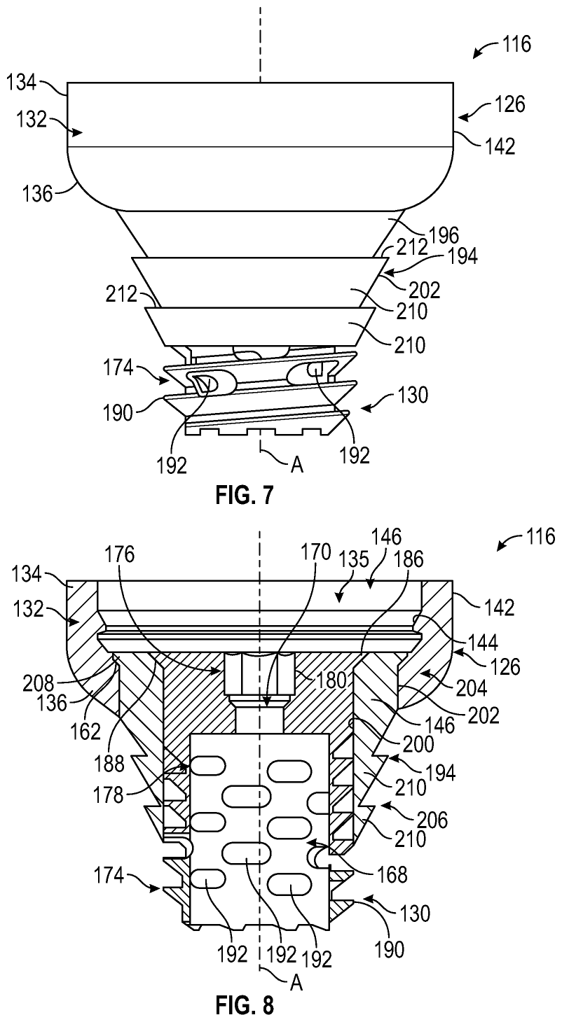

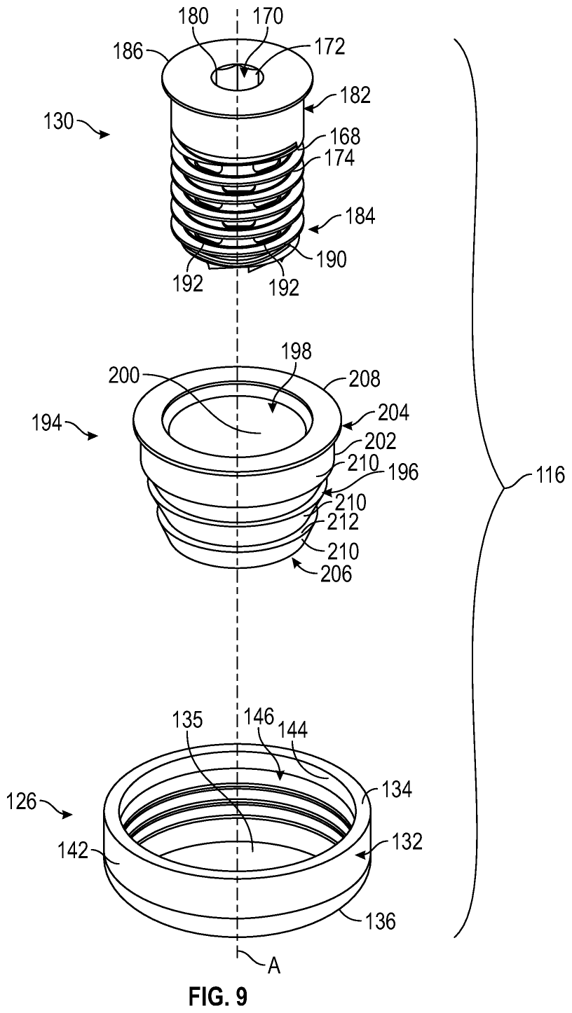

FIGS. 7, 8, and 9 illustrate another exemplary stemless implant assembly 116 that can be utilized as part of the humeral implant assembly 12 of FIGS. 1-2. In this implementation, the stemless implant assembly 116 may include a cup 126, an anti-rotation implant 194 (e.g., a first fixation device), and a screw 130 (e.g., a second fixation device). The stemless implant assembly 116 is therefore another exemplary three-piece design.

The cup 126, the anti-rotation implant 194, and the screw 130 may be coaxially arranged along a central longitudinal axis A. When the stemless implant assembly 116 is assembled/implanted, the anti-rotation implant 194 may extend at least partially through the cup 126, and the screw 130 may extend at least partially through the anti-rotation implant 194.

The cup 126 may be substantially similar in design as the cup 26 described above and may therefore include a cylindrical shaped body 132 that extends between a rim 134 located at a top or proximal side of the cylindrical shaped body 132 and a rounded base 136 located at a bottom or distal side of the cylindrical shaped body 132. The rim 134 may thus be located on an opposite side of the cylindrical shaped body 132 from the rounded base 136. Further, the cup 126 may include either a flanged design or a flangeless design at the rim 134.

The cylindrical shaped body 132 of the cup 126 may include an outer diameter wall 142 and an inner diameter wall 144. The outer diameter wall 142 may be a relatively smooth surface, and the inner diameter wall 144 may include both a smooth surface and a threaded surface.

The cylindrical shaped body 132 of the cup 126 may include a central bore 135. The central bore 135 may be a void circumscribed by the inner diameter wall 144 and may be sized and shaped to receive the anti-rotation implant 194 therethrough.

A receiving cavity 146 of the cup 126 may be configured to receive and secure either the anatomic articular implant 18A or the reverse articular implant 18B to the stemless implant assembly 116. The receiving cavity 146 may provide an inlay design in which a majority of the stemless implant assembly 116 (with the exception of the flange of the cup 126 to the extent one is provided) is disposed inside the humerus 14 post implantation. In this way, the connection between the stemless implant assembly 116 and the articular implant 18A, 18B is also inlaid rather than exhibiting an onlay design.

The anti-rotation implant 194 may include a tapered body 196 that tapers proximally to distally. The tapered body 196 may include a central bore 198. The central bore 198 may be a void circumscribed by an inner diameter wall 200 of the tapered body 196. The central bore 198 may be sized and shaped to receive the screw 130 therethrough.

The tapered body 196 of the anti-rotation implant 194 may additionally include an outer diameter wall 202 having a proximal section 204 and a distal section 206. The proximal section 204 may be a relatively smooth surface that includes a radially extending peripheral lip 208. The peripheral lip 208 may establish a proximal most portion of the proximal section 204. The peripheral lip 208 may be configured to interface with a tapered ledge 162 formed on the inner diameter wall 144 of the cup 126 to prevent over-insertion of the anti-rotation implant 194 distally through the cup 126.

The distal section 206 of the anti-rotation implant 194 may include two or more barbs 210. In an embodiment, the barbs 210 are truncated cones. Each barb includes a ledge 212. In an embodiment, the ledges 212 are circular ledges that extend circumferentially around at least a portion of the outer diameter wall 202. The ledges 212 provide a surface area for receiving backfilled fragments of bone during insertion of the anti-rotation implant 194 into the humerus 14. This is referred to as interdigitation. The interdigitation improves fixation of the anti-rotation implant 194 within the humerus 14 and thus substantially prevents rotation of the cup 126 during insertion of the screw 130.

The anti-rotation implant 194 may be pushed or malleted into the humerus 14. In an implanted position of the stemless implant assembly 116, the barbs 210 of the distal section 206 are located distally of the cup 126. At this position, the barbs 210 are capable of engaging cancellous bone 66 of the humerus 14.

The screw 130 may be substantially similar in design to the second screw 30 of FIGS. 3-5 and may include a cylindrical body 168 that includes a central bore 170. The central bore 170 may be a void circumscribed by an inner diameter wall 172 of the cylindrical body 168. The central bore 170 may include a proximal portion 176 and a distal portion 178, which may have a larger diameter than the proximal portion 176. The proximal portion 176 may include a driver recess 180 for receiving a driver instrument that can be used to insert the screw 130 through the anti-rotation implant 194.

The cylindrical body 168 of the screw 130 may additionally include an outer diameter wall 174 having a proximal section 182 and a distal section 184. The proximal section 184 may be relatively smooth and may include a radially extending peripheral lip 186. The peripheral lip 186 may establish a proximal most portion of the proximal section 182. The peripheral lip 186 may be configured to interface with a tapered ledge 188 formed on the inner diameter wall 200 of the anti-rotation implant 194 to prevent over-insertion of the screw 130 distally through the anti-rotation implant 194.

The distal section 184 of the screw 130 may include a thread 190 that is circumferentially disposed about the distal section 184 of the outer diameter wall 174. The thread 190 may be a self-tapping thread configured to allow the screw 130 to be screwed into the humerus 14 at a location that is radially inward from the anti-rotation implant 194. The thread 190 may be configured such that either a clockwise rotation or a counterclockwise rotation functions to advance the screw 130 into the humerus 14. In an implanted position of the stemless implant assembly 116, a proximal portion of the thread 190 may engage the inner dimeter wall 200 of the anti-rotation implant 194, and a distal portion of the thread 190 may be located distally of the anti-rotation implant 194. At this position, the thread 190 is capable of engaging additional cancellous bone 66 of the humerus 14 that is located radially inward of that which is engaged by the barbs 210 of the anti-rotation implant 194. The anti-rotation implant 194 and the screw 130 may therefore cooperate to provide dual fixation of the cup 126 in this implementation.

The distal section 184 of the screw 130 may additionally be equipped with a plurality of openings 192. The openings 192 may facilitate bony ingrowth post-insertion of the screw 130. The openings 192 may be oblong shaped, in an embodiment.

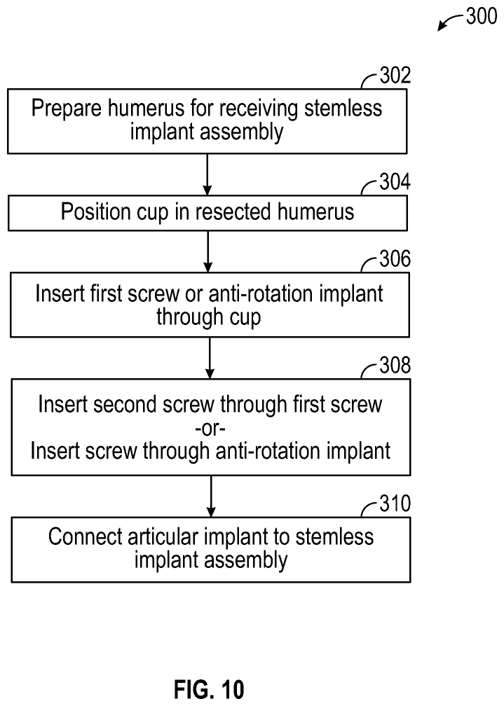

FIG. 10, with continued reference to FIGS. 1-9, schematically illustrates a surgical method 300 for performing a shoulder arthroplasty procedure. The surgical method 300 may include implanting the stemless implant assembly 16 or the stemless implant assembly 116 during the shoulder arthroplasty procedure. However, other joints could be repaired or replaced using a similar procedure to the one described below. It should further be understood that the surgical method 300 could include a greater or fewer number of steps than are specifically indicated below, and that the steps could be performed in a different order within the scope of this disclosure.

The method 300 may begin at block 302 by preparing the humerus 14 for receiving the stemless implant assembly 16 or 116. Preparing the humerus 14 may include resecting the humeral head of the humerus 14, preparing a cavity within the resected humerus for receiving the cup 26 or the cup 126, etc.

Next, at block 304, the cup 26 or the cup 126 may be positioned within the resected humerus. The cup 26 or the cup 126 may be impacted into humerus 14 until the rim 34 of the cup 26 or the rim 134 of the cup 126 is flush with the cortical rim 40.

The first screw 28 or the anti-rotation implant 194 may be inserted through the cup 26 or the cup 126, respectively, at block 306. Subsequently, at block 308, the second screw 30 may be inserted through the first screw 28, or the screw 130 may be inserted through the anti-rotation implant 194. Once implantation is complete, a combination of the first screw 28 and the second screw 30 or a combination of the anti-rotation implant 194 and the screw 130 may engage the cancellous bone 66 of the humerus 14 for providing strong fixation.

Finally, at block 310, an articular implant 18A, 18B may be connected to the implanted stemless implant assembly 16 or stemless implant assembly 116. The stemless implant assembly 16 or the stemless implant assembly 116 therefore establishes a convertible platform for interfacing with either anatomic articular implants or reverse articular implants.

The exemplary arthroplasty implant systems of this disclosure employ stemless humeral implant assemblies that include cups that are fixated by at least one screw. The assemblies are capable of establishing a convertible platform for interfacing with articular implants. The stemless implants may provide relatively strong fixation and may further provide inlaid designs that allow for inlay reverse prothesis configurations.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A stemless implant assembly for a humeral implant assembly, comprising:
a cup;
a first fixation device insertable through a first central bore of the cup; and
a second fixation device insertable through a second central bore of the first fixation device.

2. The stemless implant assembly as recited in claim 1, wherein the first fixation device is a major screw, and the second fixation device is a minor screw.

3. The stemless implant assembly as recited in claim 1, wherein the first fixation device is an anti-rotation implant, and the second fixation device is a screw.

4. The stemless implant assembly as recited in claim 1, wherein, in an assembled condition of the stemless implant assembly, a first distal section of the first fixation device is a first distance from the cup, and a second distal section of the second fixation device is a second distance from the cup, and further wherein the second distance is greater than the first distance.

5. The stemless implant assembly as recited in claim 4, wherein the second distal section includes a thread.

6. The stemless implant assembly as recited in claim 1, wherein the first central bore extends between a rim and a rounded base of the cup.

7. The stemless implant assembly as recited in claim 6, wherein the rim includes a flange.

8. The stemless implant assembly as recited in claim 1, wherein the first fixation device includes a thread or a barb.

9. The stemless implant assembly as recited in claim 8, wherein the second fixation device includes a thread.

10. The stemless implant assembly as recited in claim 1, wherein the cup includes a cylindrical shaped body having a threaded inner diameter wall and a smooth outer diameter wall.

11. A humeral implant assembly for an arthroplasty implant system, comprising:
an articular implant; and
a stemless implant assembly adapted to establish a convertible platform for receiving the articular implant;
wherein the stemless implant assembly includes a cup, a first fixation device insertable through the cup, and a second fixation device insertable through the first fixation device.

12. The humeral implant assembly as recited in claim 11, wherein the articular implant includes a concave articular surface.

13. The humeral implant assembly as recited in claim 11, wherein the articular implant includes a convex articular surface.

14. The humeral implant assembly as recited in claim 11, wherein the cup includes a cylindrical shaped body having a threaded inner diameter wall and a smooth outer diameter wall.

15. The humeral implant assembly as recited in claim 11, wherein the first fixation device is a screw that includes an outer diameter wall and a thread circumferentially disposed about at least a portion of the outer diameter wall.

16. The humeral implant assembly as recited in claim 11, wherein the first fixation device is an anti-rotation implant that includes a tapered body having at least two barbs, and further wherein each of the at least two barbs includes a ledge extending circumferentially around at least a portion of the tapered body.

17. The humeral implant assembly as recited in claim 11, wherein the second fixation device is a screw that includes an outer diameter wall and a thread circumferentially disposed about at least a portion of the outer diameter wall.

18. The humeral implant assembly as recited in claim 11, wherein, in an assembled condition of the stemless implant assembly, a first distal section of the first fixation device is a first distance from the cup, and a second distal section of the second fixation device is a second distance from the cup, and further wherein the second distance is greater than the first distance.

19. The humeral implant assembly as recited in claim 11, wherein the cup, the first fixation device, and the second fixation device are coaxially arranged along a central longitudinal axis of the stemless implant assembly.

20. A surgical method, comprising:
preparing a humerus for receiving a cup of a stemless implant assembly;
positioning the cup within the prepared humerus;
inserting a first fixation device of the stemless implant assembly through the cup and into a cancellous bone of the humerus; and
inserting a second fixation device of the stemless implant assembly through the first fixation device and into the cancellous bone,
wherein the second fixation device extends further into the cancellous bone than the first fixation device after the inserting.
